(19) United States
(12) Patent Application Publication
(10) Pub. No.: US 2016/0030293 A1
(43) Pub. Date: Feb. 4, 2016

(54) Vented Connector for Feeding Syringe

(71) Applicant: Covidien LP, Mansfield, MA (US)

(72) Inventors: Michael Dorsey, Edwardsville, IL (US); James Harr, Wentzville, MO (US)

(21) Appl. No.: 14/813,853

(22) Filed: Jul. 30, 2015

Related U.S. Application Data
(60) Provisional application No. 62/030,945, filed on Jul. 30, 2014.

(51) Int. Cl. A61J 15/00; A61M 5/34; A61M 5/38; A61M 39/12

(52) U.S. Cl. CPC A61J 15/0096; A61J 15/0003; A61M 39/12; A61M 5/347; A61J 15/0026; A61M 5/38; A61M 2039/0009

(57) ABSTRACT

An enteral feeding connector includes a connector body that can be removably attached to a tip of a syringe, which has a component that makes a fluid tight seal with the syringe tip. An air venting stem integrally formed with the component can extend into a barrel of the syringe. An air venting passage extends through the air venting stem and opens in the barrel of the syringe. An enteral fluid passage extends through the component and is in fluid communication with the barrel of the syringe.

### CROSS-REFERENCE TO RELATED APPLICATION

This application claims the benefit of priority to U.S. Patent Application No. 62/030,945, titled VENTED CONNECTOR FOR FEEDING SYRINGE, filed on Jul. 30, 2014, which is incorporated herein by reference in its entirety for all purposes.

### FIELD OF THE INVENTION

The present invention generally relates to an enteral feeding administration set including an enteral feeding connector.

### BACKGROUND

There are different ways of enterally administrating liquid nutrient and/or liquid medicine to a patient. One way of enteral feeding is by gravity. A feeding tube, such as a nasogastric administration tube, running from a container of liquid nutrients and/or medicine is inserted into the stomach of the patient, and the liquid flows from the container, by gravity, into the stomach of the patient. Thompson, in U.S. Pat. No. 4,623,343, disclosed a parenteral fluid administration apparatus and method, which is provided with an air vent but has a ball valve that prevents fluid from exiting the air channel.

### SUMMARY

There is disclosed an enteral feeding connector for use with a syringe having barrel and a female tip defining a tip passage in communication with the barrel. The connector can comprise a connector body including a syringe-connecting portion adapted to connect to and fluidly seal the female tip of the syringe, a tubing-connecting portion adapted to connect to tubing, an air venting stem extending outward from the connector body, the air venting stem configured to extend into the barrel when the connector body is connected to the female tip of syringe, an air venting passage extending through the air venting stem and the connector body, the air venting passage configured to fluidly connect an interior of the barrel with atmosphere when the connector body is connected to the female tip of the syringe, and an enteral fluid passage, separate from the air venting passage, extending through the connector body, the enteral fluid passage fluidly connecting the interior of the barrel with the tube-connecting portion. The connector body, in some cases, further comprises a male component sized and shaped for reception in the tip passage to make sealing connection with female tip. The enteral feeding connector, in some cases, further comprising an inlet in the connector body, fluidly connecting the interior of the barrel with the tube-connecting portion through the enteral fluid passage. The enteral feeding connector, in some cases further comprising a membrane which is air permeable and liquid impervious, the membrane mounted on the connector body and over an open end of the air venting passage. The membrane, in some cases, is releasably attachable to the connector body. The membrane can be comprised of an oleophobic and hydrophobic material. The enteral feeding connector, in some cases, further comprises a filter disposed over the air venting passage, the filter comprising a hydrophobic and oleophobic material. In some cases, the enteral feeding connector does not a valve that inhibits exit of fluid from the barrel through the air venting passage.

There is also disclosed a method of delivering a liquid to a neonate, comprising disposing the liquid to be delivered to the neonate into an interior of a barrel of a syringe having a plunger slidably engageable therewith; and connecting a enteral feeding connector to a tip of the syringe, the tip having a tip passage in communication with the barrel, the connector comprising a connector body including a syringe-connecting portion adapted to removably connect to the tip of the syringe, and a tubing-connecting portion adapted to connect to tubing, wherein the syringe-connection portion includes a male component adapted to form a seal with the tip of the syringe when the connector body is connected to the tip of the syringe, an air venting stem extending outward from the connector, the air venting stem configured to extend into the barrel when the connector body is connected to the syringe, an air venting passage extending through the air venting stem and the component, the air venting passage configured to fluidly connect the barrel with atmosphere when the connector body is connected to the tip of the syringe, and an enteral fluid passage, separate from the air venting passage, extending through the connector body, the enteral fluid passage fluidly connecting an interior of the barrel with the tube-connecting portion. The method, in some cases, further comprises disposing a filter disposed over the air venting passage, the filter comprising a hydrophobic and oleophobic material.

Other features will be in part apparent and in part pointed out hereinafter.

### BRIEF DESCRIPTION OF THE DRAWINGS

FIG. 1 is a perspective view of an enteral feeding set;

FIG. 2 is an exploded perspective view of the enteral feeding set;

FIG. 3 is an enlarged sectional view of an enteral feeding connector of the enteral feeding set, a filter of the enteral feeding connector being removed for illustrative purposes;

FIG. 4 is an enlarged sectional view of the enteral feeding set;

FIGS. 5A-E are schematic illustrations of another embodiment of an enteral feeding connector, with FIG. 5A showing a perspective view of the connector, FIG. 5B showing an expanded view of the connector of FIG. 5A, FIG. 5C showing a side elevational view of the connector of FIG. 5A, FIG. 5D showing a top plan view of the connector of FIG. 5A, and FIG. 5E showing a cross-sectional view of the connector of FIG. 5A along section E-E of FIG. 5D.

Corresponding reference characters indicate corresponding parts throughout the drawings.

### DETAILED DESCRIPTION

In one aspect, an enteral feeding connector for use with a syringe having barrel and a female tip defining a tip passage in communication with the barrel is disclosed. The connector generally comprises a connector body including a syringe-connecting portion adapted to removably connect to a tip of a syringe, and a tubing-connecting portion adapted to connect to tubing. The syringe-connection portion includes a male component adapted to form a fluid tight seal with the female tip of the syringe when inserted into the tip passage of the syringe. An air venting stem is integrally formed with the male component and extends outward therefrom. The air venting stem is configured to extend into the barrel when the male component is inserted into the tip passage of the syringe. An air venting passage extends through the air venting stem and the male component. The air venting passage is configured to fluidly connect the barrel with atmosphere when the male component is inserted into the tip passage of the syringe. An enteral fluid passage, separate from the air venting passage, extends through the male component to the tubing-connecting portion of the connector body. The enteral fluid passage is configured to deliver enteral fluid from the syringe to the tubing. The enteral fluid passage can be configured to fluidly connect an interior volume of the barrel with the tubing-connecting portion. In some cases, the component is sized and shaped for reception in the tip passage to make sealing connection with the syringe in the tip passage. In some cases, the enteral fluid passage opens laterally of the component. In some cases, the enteral feeding connector further comprises a membrane which is air permeable and liquid impervious, wherein the membrane is mounted on the connector body and closing an open end of the air venting passage. In some cases, the membrane is releasably attachable to the connector body. In some cases, the enteral feeding connector is free of valves. In some cases, the enteral feeding connector further comprises a filter disposed on the air venting passage, wherein the filter comprises a hydrophobic material. In some cases, the enteral feeding connector further comprises a drip chamber fluidly connected to the tubing-connecting portion.

In another aspect, a method of delivering a liquid to a neonate comprises disposing the liquid to be delivered to the neonate into an interior of a barrel of a syringe having a plunger slidably engage therewith; and connecting a enteral feeding connector to a tip of the syringe, the tip having a tip passage in communication with the barrel, the connector comprising a connector body including a syringe-connecting portion adapted to removably connect to the tip of the syringe, and a tubing-connecting portion adapted to connect to tubing, wherein the syringe-connection portion includes a component adapted to form a fluid tight seal with the tip of the syringe when the connector body is connected to the tip of the syringe, an air venting stem integrally formed with the component and extending outward therefrom, the air venting stem configured to extend into the barrel when the connector body is connected to the syringe, an air venting passage extending through the air venting stem and the component, the air venting passage configured to fluidly connect the barrel with atmosphere when the connector body is connected to the tip of the syringe, and an enteral fluid passage, separate from the air venting passage, extending through the component to the tubing-connecting portion of the connector body, the enteral fluid passage fluidly connecting an interior of the barrel with the tube-connecting portion. In some cases, the method further comprises disposing a filter over the air venting passage, the filter comprising a hydrophobic material. In some cases, the method further comprises fluidly connecting a drip chamber fluidly to the tubing-connecting portion.

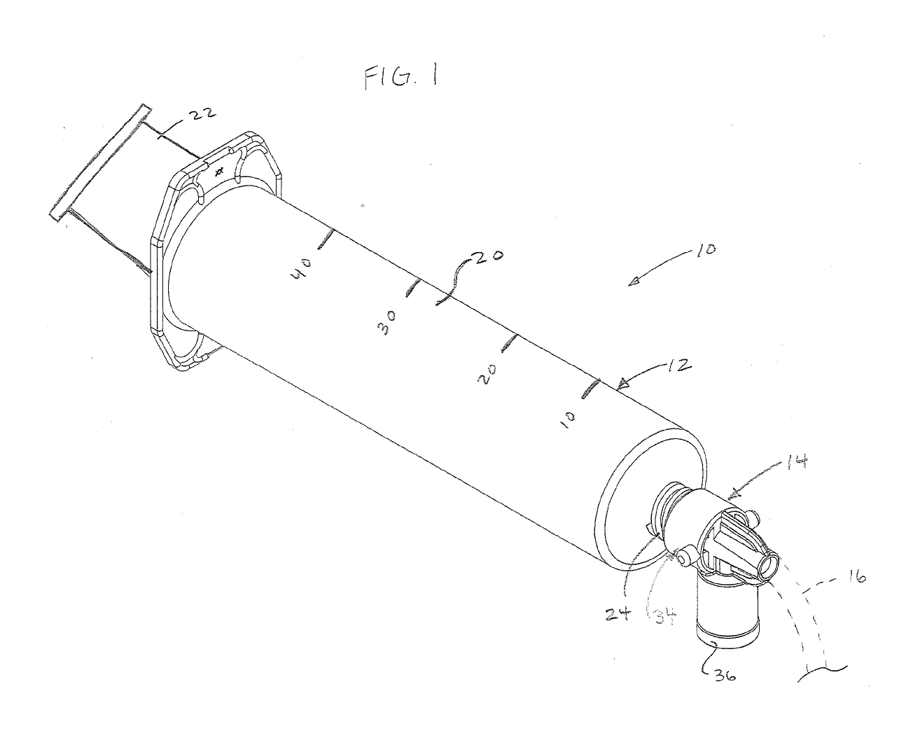

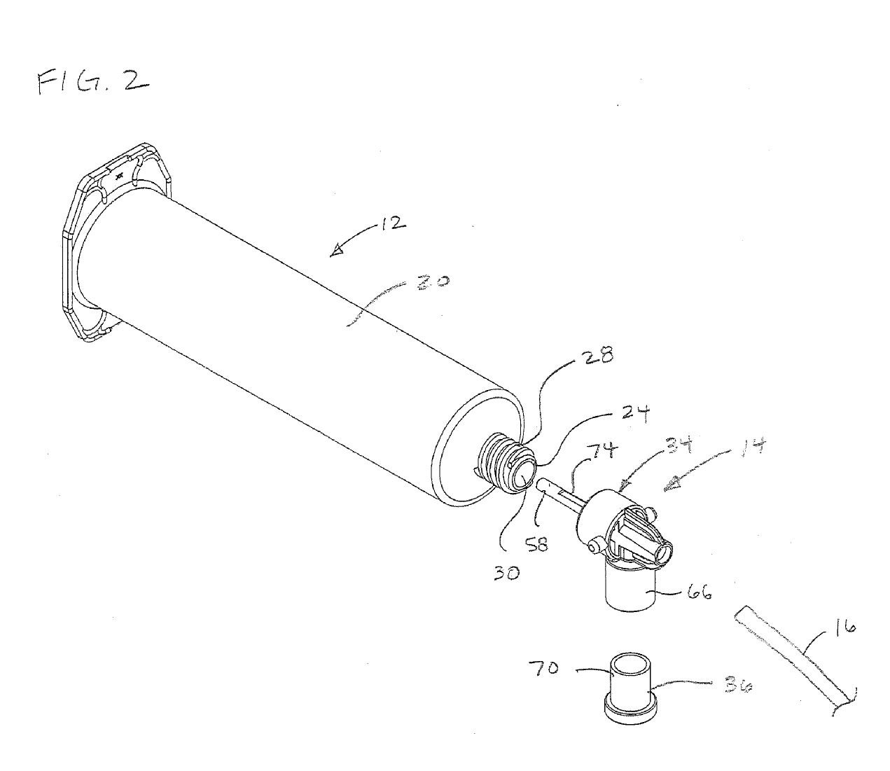

Referring to FIGS. 1, 2 and 5A-5E, an enteral feeding administration set or enteral feeding set is generally indicated at reference numeral 10. The enteral feeding administration set includes a syringe, generally indicated at reference numeral 12, and an enteral feeding connector, generally indicated at reference numeral 14, for fluidly connecting the syringe to feeding tubing 16. The syringe 12 may be a conventional syringe including a barrel 20, which may be graduated, and a plunger 22 slidably received in the barrel. In the illustrated embodiment, the syringe 12 includes a female tip 24 including an external thread 28 and defining a tip passage 30 in communication with the interior of the barrel 20. The syringe 12 may be of other configurations without departing from the scope of the present invention.

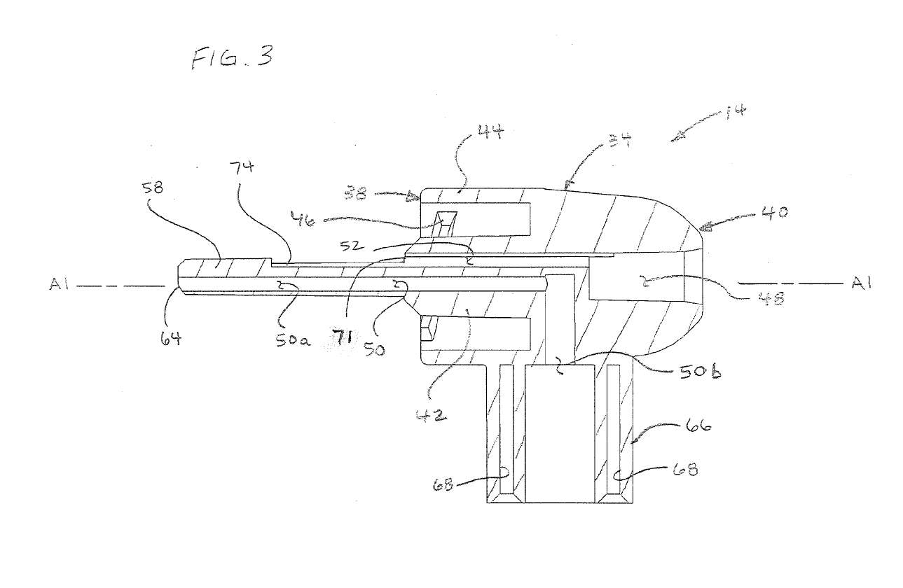

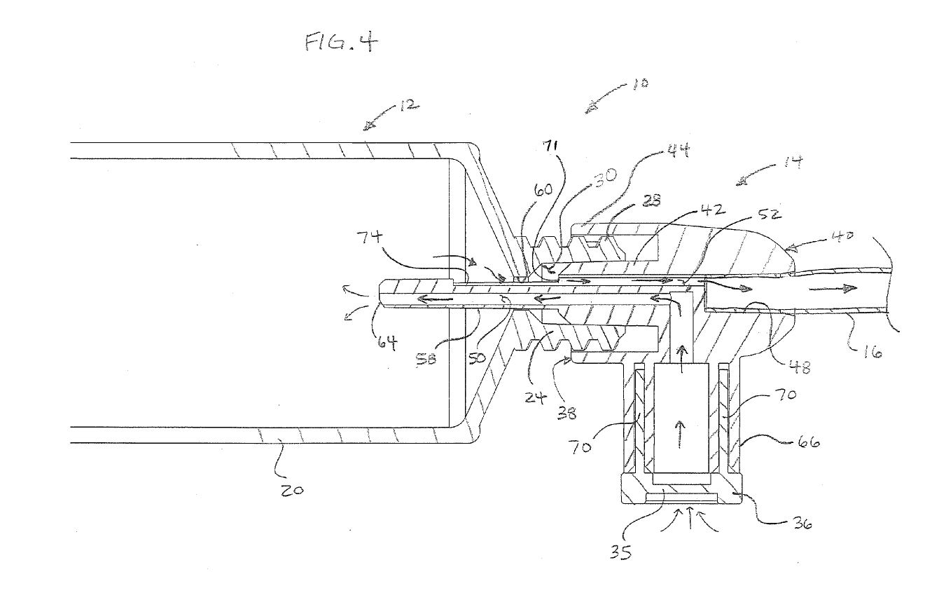

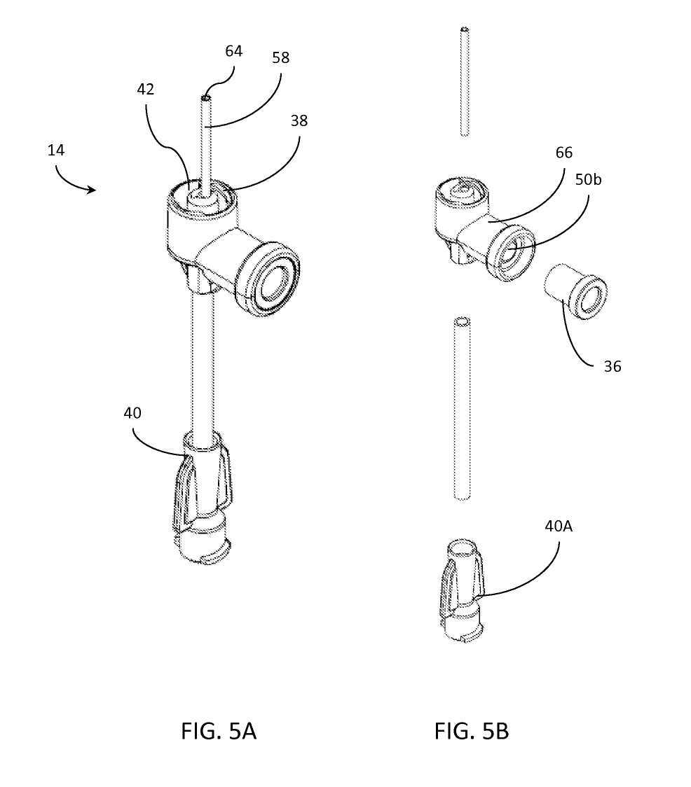

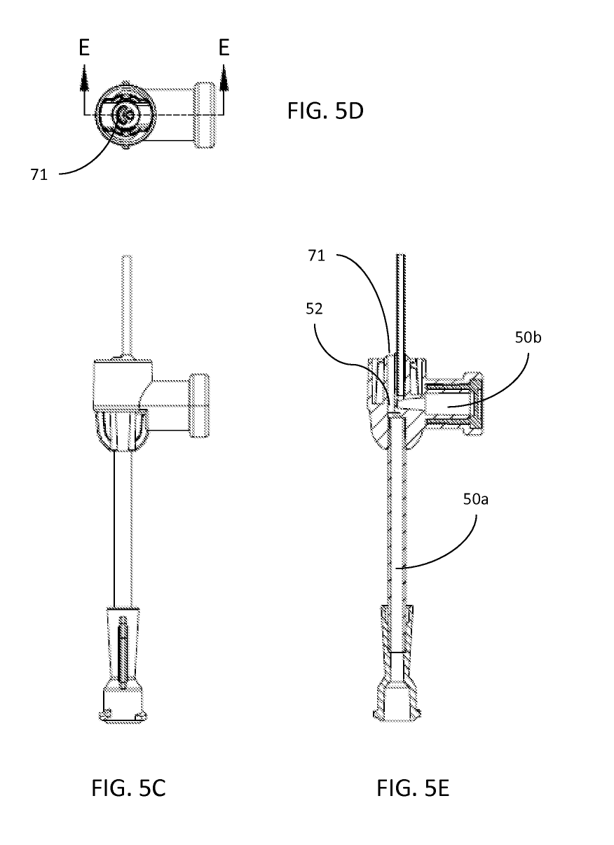

The enteral feeding connector 14 comprises a one-piece, integrally formed, molded connector body, generally indicated 34, and an air filter 36 including an air-permeable, liquid-impermeable membrane 35 (FIG. 4) secured to the connector body, as explained in more detail below. Referring to FIG. 3, the connector body 34 includes a syringe-connecting portion, generally indicated at 38, adapted to removably connect to the female tip 24 of the syringe 12, and a tubing-connecting portion, generally indicated at 40, adapted to connect (e.g., removably connect) to the feeding tubing 16. In some cases, as shown in FIGS. 5A-5E, the connector includes a male connector 40A configured to conform with the ISO 80369-3 standard. The syringe-connecting portion 38 includes a male component 42 configured to form a liquid-tight seal with the female tip 24 of the syringe 12 when inserted into the tip passage 30. An outer skirt 44 of the syringe-connecting portion 38 surrounds the male component 42 and includes an internal thread 46 configured to threadably mate with the external thread 28 of the female tip 24. The female tip can be a threaded female tip. The tubing-connecting portion 40 defines a cavity 48 for receiving the feeding tubing 16. The feeding tubing 16 may be secured within the cavity 48, using adhesive for example, or in other ways. For example, the tubing connecting portion may be a protrusion (not shown) sized to be received within an interior of the feeding tubing. In some cases, the syringe-connecting portion 38 is configured to conform with the ISO 80369-3 standard.

Referring to FIG. 3, the enteral feeding connector 14 defines separate air venting and enteral fluid passages 50, 52, respectively. The air venting passage 50 fluidly connects the interior of the barrel 20 to atmosphere to vent the interior of the barrel, as explained in more detail below. An air venting stem 58 extends axially outward from the male component 42 of the syringe-connecting portion 38 and defines a portion of the air venting passage 50. As shown in FIG. 4, when the connector 14 is secured to the syringe 12, the air venting stem 58 extends through a neck 60 of the syringe (located between the tip passage 30 and the interior of the barrel 20) and into the interior of the barrel such that an outlet 64 of the air venting passage 50 is received in and fluidly connected to the interior of the barrel. As shown in FIG. 3, the air venting passage 50 has an axial portion 50a extending generally axially along an axis Al of connector body 34, through the air venting stem 58 and the male component 42. An orthogonal portion 50b of the air venting passage leading to the air filter 36 extends generally orthogonal to the axial portion 50a and the axis Al of the connector body. A part of the orthogonal portion 50b is defined by an air filter adaptor 66 configured to removable attach the air filter 36 to the connector body 34. The air filter adaptor 66 defines an annular cavity 68 that is adapted to receive a cylindrical extension 70 of the air filter 36, as shown in FIGS. 3 and 4.

Referring to FIG. 3, the enteral fluid passage 52 fluidly connects the syringe 12 to the feeding tubing 16. The enteral fluid passage 52 extends generally axially through the male component 40 of the syringe-connecting portion 38, generally adjacent and parallel to the air venting passage 50, to the cavity 48 of the tubing-connecting portion 40. When the connector 14 is secured to the syringe 12, an inlet 71 of the enteral fluid passage is received in and in communication with the tip passage 30. The air venting stem 58 is configured to allow fluid in the barrel 20 to flow through the neck 60 of the syringe 12 and into the tip passage 30 so that the fluid can enter the inlet 71 of the enteral fluid passage 52. As illustrated in FIGS. 5D and 5E, the inlet 71 can be kidney shaped opening in the connector body to allow as large an opening as possible for fluid connection into fluid passage 52. In the illustrated embodiment, the air venting stem 58 defines a furrow or groove 74 extending along its exterior surface to allow fluid to flow through the neck 60 of the syringe 12. The groove 74 may be considered an extension of the enteral fluid passage 52 and as a result, the passage opens laterally outwardly of the male component 42. Other ways of providing clearance for fluid to flow through the neck 60 of the syringe 12 do not depart from the scope of the present invention.

The membrane can exhibit oleophobic and hydrophobic characteristics and in some cases can include a plurality of layers of glass fiber media. A commercially available material suitable as the membrane includes PALLFLEX B070DW cellulose supported microglass fiber membrane from Pall Corporation. The use of a material having both oleophobic and hydrophobic characteristics advantageously prevents or at least inhibits passage of liquid such as the feeding fluid contained in the syringe barrel while allowing air to pass therethrough, which avoids the use of further components, such as check valves, e.g., a ball valve, that would be intended to fluidly isolate the membrane from the feeding fluid.

The above disclosed enteral feeding set 10 may be used for enteral feeding of neonates by gravity feeding. In such a method, the enteral liquid is drawn into the syringe 12 by pulling back on the plunger 22. The amount of enteral liquid may be measured using graduation markings on the barrel 20 of the syringe 12. This provides accurate delivery of small amounts of enteral liquid to the neonate, which is important for neonate health. After filling the syringe 12 with the proper amount of enteral liquid, the enteral feeding connector 14 may be attached to the syringe tip 24, such as by threading the syringe connection portion 38 onto the tip. The feeding tubing 16 may be pre-attached to the enteral feeding connector 14 or the tube may be attached to the connector after securing the connector to the syringe 12. Moreover, the tube 16 may be further attached to a feeding tube (e.g., a nasogastric feeding tube), which is placed within the digestive system of the neonate, before or after fluidly connecting the syringe 12 thereto.

Without removing the plunger 22, the syringe 12 is placed in an elevated position with the tip 24 facing downward. Initially, the entire air venting stem 58 in the barrel 20 may be under the feeding solution. Liquid feeding solution may enter the air venting passage 50, but will be blocked from leaking out of the connector 14 by the membrane 35, which may be air permeable, but liquid impermeable. As the feeding solution is delivered to the neonate, the level within the barrel 20 eventually drops below the upper end of the air venting stem 58. As will be understood, the dropping of the liquid level in the barrel 20 also creates a vacuum pressure above the feeding solution. Once the outlet 64 of the air venting stem 58 is exposed, air passes through the membrane 35, forcing any liquid in the air venting passage 50 to be forced out into the barrel for delivery to the neonate. After liquid is expelled, air flows into the barrel 20 above the feeding solution, relieving the vacuum pressure. Because the interior chamber of the barrel 20 is vented, the enteral liquid in the barrel 20 of the syringe 12 flows freely through the neck 60 of the syringe and into the enteral fluid passage 52. From the enteral fluid passage 52, the enteral fluid flows through the tubing 16 and into the feeding tube. The method may further include the use of a drip chamber and/or a roller clamp to interrupt fluid flow (not shown). The foregoing functionality is achieved without the use of valves.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

1. An enteral feeding connector for use with a syringe having barrel and a female tip defining a tip passage in communication with the barrel, the connector comprising:
a connector body including
a syringe-connecting portion adapted to connect to and fluidly seal the female tip of the syringe, a tubing-connecting portion adapted to connect to tubing,
an air venting stem extending outward from the connector body, the air venting stem configured to extend into the barrel when the connector body is connected to the female tip of syringe,
an air venting passage extending through the air venting stem and the connector body, the air venting passage configured to fluidly connect an interior of the barrel with atmosphere when the connector body is connected to the female tip of the syringe, and
an enteral fluid passage, separate from the air venting passage, extending through the connector body, the enteral fluid passage fluidly connecting the interior of the barrel with the tube-connecting portion.

2. The enteral feeding connector as set forth in claim 1 wherein the connector body further comprises a male component sized and shaped for reception in the tip passage to make sealing connection with female tip.

3. The enteral feeding connector as set forth in claim 1, further comprising an inlet in the connector body, fluidly connecting the interior of the barrel with the tube-connecting portion through the enteral fluid passage.

4. The enteral feeding connector as set forth in claim 1, further comprising a membrane which is air permeable and liquid impervious, the membrane mounted on the connector body and over an open end of the air venting passage.

5. The enteral feeding connector as set forth in claim 4 wherein the membrane is releasably attachable to the connector body.

6. The enteral feeding connector as set forth in claim 4, wherein the membrane is comprised of an oleophobic and hydrophobic material.

7. The enteral feeding connector as set forth in claim 1, further comprising a filter disposed over the air venting passage, the filter comprising a hydrophobic and oleophobic material.

8. The enteral feeding connector as set forth in claim 1, without having a valve that inhibits exit of fluid from the barrel through the air venting passage.

9. A method of delivering a liquid to a neonate, comprising:
disposing the liquid to be delivered to the neonate into an interior of a barrel of a syringe having a plunger slidably engageable therewith; and
connecting a enteral feeding connector to a tip of the syringe, the tip having a tip passage in communication with the barrel, the connector comprising a connector body including a syringe-connecting portion adapted to removably connect to the tip of the syringe, and a tubing-connecting portion adapted to connect to tubing, wherein the syringe-connection portion includes a male component adapted to form a seal with the tip of the syringe when the connector body is connected to the tip of the syringe, an air venting stem extending outward from the connector, the air venting stem configured to extend into the barrel when the connector body is connected to the syringe, an air venting passage extending through the air venting stem and the component, the air venting passage configured to fluidly connect the barrel with atmosphere when the connector body is connected to the tip of the syringe, and an enteral fluid passage, separate from the air venting passage, extending through the connector body, the enteral fluid passage fluidly connecting an interior of the barrel with the tube-connecting portion.

10. The method as set forth in claim 9, further comprising disposing a filter disposed over the air venting passage, the filter comprising a hydrophobic and oleophobic material.

11. An enteral feeding system comprising:
a syringe having barrel and a female tip defining a tip passage in communication with the barrel;
a connector having a connector body including a syringe-connecting portion adapted to connect to and fluidly seal the female tip of the syringe, a tubing-connecting portion adapted to connect to tubing, an air venting stem extending outward from the connector body, the air venting stem configured to extend into the barrel when the connector body is connected to the female tip of syringe, an air venting passage extending through the air venting stem and the connector body, the air venting passage configured to fluidly connect an interior of the barrel with atmosphere when the connector body is connected to the female tip of the syringe, a means for inhibiting exit of liquid contained within the barrel through the air venting passage, and an enteral fluid passage, separate from the air venting passage, extending through the connector body, the enteral fluid passage fluidly connecting the interior of the barrel with the tube-connecting portion.
